(19) United States
(12) Patent Application Publication
(10) Pub. No.: US 2016/0287528 A1
(43) Pub. Date: Oct. 6, 2016

(54) METHODS AND COMPOSITIONS FOR USE IN AQUACULTURE

(71) Applicant: Avivagen Inc., Ottawa (CA)

(72) Inventors: Larry Arthur STOBBS, Pendleton, IN (US); James Gary NICKERSON, Charlottetown (CA); Clayton Paul DICK, Guelph (CA)

(21) Appl. No.: 15/154,025

(22) Filed: May 13, 2016

Related U.S. Application Data
(63) Continuation of application No. 13/579,813, filed on Feb. 28, 2013, now abandoned.
(60) Provisional application No. 61/306,125, filed on Feb. 19, 2010.

(51) Int. Cl. A61K 31/015; A61K 9/00; A61K 9/107; A23K 50/80; A61K 31/00; A61K 9/50; A23K 20/179; A61K 9/14; A61K 45/06

(52) U.S. Cl. CPC A61K 31/015; A61K 9/14; A61K 9/0056; A61K 9/1075; A61K 45/06; A61K 31/00; A61K 9/50; A61K 9/5057; A61K 9/5015; A61K 9/5036; A61K 9/007; A23K 20/179; A23K 50/80

(57) ABSTRACT

The invention features compositions, methods, and kits for the administration of an oxidatively transformed carotenoid, or a fractionated component thereof, for use in aquaculture.

### CROSS-REFERENCE TO RELATED APPLICATION

This application claims benefit of U.S. Provisional Application No. 61/306,125, filed Feb. 19, 2010, which is hereby incorporated by reference.

### BACKGROUND OF THE INVENTION

The invention relates to the use of oxidatively transformed carotenoid in aquaculture.

The annual worldwide production of crustaceans is over 8 million metric tons. Of this figure over half is made up of shrimp and prawns and the proportion of this production coming from farms has increased rapidly since the 1980s. See Food and Agriculture Organization of the United Nations yearbook, Fishery statistics, Aquaculture production 2000, vol. 90/2. Aquaculture farming carries the risk of financial losses due to disease, either through mortality or reduced meat quality, resulting in reduced profit margins. There is an extensive literature, dating back some thirty years, on the problem of disease in cultured shellfish (Anderson et al., Bulletin of the Office of International Epizootiology 69:1239 (1968); Fisher et al., Proceedings of the World Mariculture Society 7:511 (1976); and Sindermann C. J., Helgolander Meeresuntersuchungen 37:505 (1984)). The economic cost of this to the aquaculture industry can be considerable. Many shrimp farms around the world have been particularly badly hit by epidemics of White Spot, Taura or Yellow Head Virus (Krishna et al., World Aquaculture 28:14 (1997); Flegel et al., Journal of Applied Icthyology 14:269 (1998); Lightner D. V., Journal of Applied Aquaculture 9:27 (1999); and Nair M. R., World Aquaculture 31:10 (2000)). It is notoriously difficult to estimate effects of shrimp disease since a common strategy is to organize emergency harvests at the first sign of disease but reportedly in Central and South America, shrimp production fell by c. 17% during the period 1998-1999, mainly as a result of viral infections (Rosenberry B., editor. World shrimp farming 1999. San Diego (USA): Shrimp News International; 1999). A survey of the shrimp aquaculture industry in Thailand found that approximately 66% of farmers had experienced at least one disease outbreak per year producing a financial loss of over $6,000 per hectare per year. The losses due to viral diseases in the Chinese shrimp industry were estimated in 1993 to stand at 1 billion dollars. In all of Asia losses were estimated in 1995 to amount to 3 billion dollars per year. See Subasinghe R., Fish health and quarantine. In: Review of the state of world aquaculture. FAO Fisheries Circular No. 866, Rev. 1. FAO, Rome, 1997. 163 pp. It is clear that this problem is severe, a fact which has been acknowledged by the World Bank which recommended that an investment of 275 million dollars should be made in shrimp disease research. See Lundin C. G., Marine/Environmental Paper No. 4. Land, Water and Natural Habitats Division, Environment Department, The World Bank; 1996. 45 pp.

A large proportion of shellfish aquaculture is dependent on wild caught broodstock that may be netted from the wild with pre-existing bacterial or viral infections. Aquaculture practices themselves may further exacerbate the problem because stock animals are often kept under stressful conditions of overcrowding, high food levels, elevated water temperature and poor water quality (Lee D. C., Wickins J. F., Crustacean farming: Blackwell Scientific Publications; 1992 392 pp.). In these stressful environments diseases associated with opportunistic bacteria, such as Vibrio spp. or Pseudomonas spp. (Sindermann C. J., Lightner D. V., Disease diagnosis and control in North American marine aquaculture 2^{nd} edn, revised Amsterdam: Elsevier; 1988 431 pp.), can become prevalent. This can compound the problem associated with more pathogenic organisms and is often further worsened by the repeated restocking of cages and ponds, leading to the accumulation of pathogens and opportunistic bacteria in the water and sediment adjacent to the farm. The potential for a disease outbreak poses a continual threat to the existence of any shellfish farm or hatchery, and once an infection occurs it can prove devastating to the entire stock.

The application of antibiotics or other chemicals to culture ponds is expensive and undesirable as it risks contamination of both the environment and the final product (Capone et al., Aquaculture 145:55 (1996); Collier et al., Journal of Experimental Marine Biology and Ecology 230:131 (1998); and Grant et al., Mar Pollut Bull 36:566 (1998)), as well as risk of mortality and/or impaired growth in juvenile stock (Swastika et al., Bulletin of the Brackishwater Aquaculture Development Centre, Jepara 9:56 (1992)). The repeated application of antibiotics, in the long term, is also encouraging the spread of drug resistant pathogens (Brown J. H., World Aquaculture 20:34 (1989); Juwana S., Oseana 15:93 (1990); Karunasagar et al., Aquaculture 28:203 (1994); and Smith et al., Ann Rev Fish Dis 4:273 (1994)) and this practice, at least in Europe, is being phased out. Moreover, chemical disinfection may be incompatible with geographical location of the shellfish farm or with the physical requirements of the stock. There is a very great need to maximize the immunocompetence of the stock while minimizing the use of therapeutic chemicals (see Bachere et al., Fish Shellfish Immunol 5:597 (1995)). It is not surprising, therefore, that there has been growing interest in finding ways to protect stock prophylactically in a manner conceptually equivalent to the use of vaccines now routine for humans and agricultural livestock.

Similar problems exist in the farming of finfish, whether for consumption (e.g., catfish, trout, salmon, and tilapia) or ornamental fish (e.g. fresh, brackish, and salt water tropical fish). Although a hatchery or aquarium setting allows for the use of established fish aquaculture technology and proven hatchery methods (e.g., well-aerated oxygen-rich water within a controlled temperature range, population control, and protection from predators), such fish are still subjected on a daily basis to numerous physical stressors which can have a detrimental impact on their health. Such physical stressors include the environmental disturbances caused by normal fish hatchery operations, such as moving, netting, pumping, crowding, cleaning, water-changing, sampling, counting, tagging, fin-clipping, transporting, and stocking fish; all of which are physical stressors which can adversely affect otherwise-healthy fish. This is evidenced by the increased fish mortality that often occurs at times such physical stressors are imposed, even during the most carefully carried out aquaculture operations.

There is a need to control, prevent, or minimize the devastating effects of disease and increased mortality associated with finfish and shellfish aquaculture.

### SUMMARY OF THE INVENTION

The invention provides compositions, methods, and kits for the use of oxidatively transformed carotenoid and components thereof. The compositions can be useful for aquaculture, for example, in the farming/ranching of shellfish and finfish.

In a first aspect the invention features a method of ameliorating an effect of physical stress in a fish (e.g., trout) by administering to the fish a composition including oxidatively transformed carotenoid, or a fractionated component thereof, in an amount sufficient to ameliorate the effect of physical stress, wherein the physical stress arises from overcrowding or from an environmental change. In certain embodiments, the physical stress arises from changes in salinity, temperature, pH, oxidative stress, or exposure to a chemotherapeutic agent. The fish can be selected from catfish, carp, trout, salmon, char, whitefish, sturgeon, tench, roach, pike, pike-perch, sole, turbot, yellowtail, bass, milkfish, tilapia, walleye, gray mullet, eels, angel fish, barb, catfish, cichlids, corydoras, danio, discus, gourami, guppy, koi, loach, minnow, molly, platy, plecostumas, rainbow and platy variatus, rasbora, shark, sword, tetra, botia, knife fish, lionfish, brackish-archer fish, flounder, golby, half beak, mono, needle fish, pipe fish, puffer, scat, bumble bee, twin spot damsel, yellowtail damsel, barbed squirrel, wrasse, black-spotted puffer, trigger fish, puffer, butterfly fish, and any other fish described herein.

In a related aspect, the invention features a method of ameliorating an effect of physical stress in a shellfish by administering to the shellfish a composition including oxidatively transformed carotenoid, or a fractionated component thereof, in an amount sufficient to ameliorate the effect of physical stress. In certain embodiments, the physical stress arises from overcrowding or from an environmental change (e.g., changes in salinity, temperature, pH, oxidative stress, or exposure to a chemotherapeutic agent).

In any of the above aspects, the effect of physical stress can be concomitant with aggression, concomitant with a decrease in meat quality or marketability, and/or concomitant with an increase in mortality.

In particular embodiments of the above aspects, the effect of physical stress is not the result of an inflammatory condition or infection.

The invention features a method of treating a shellfish having, or at risk of an infection by administering to the shellfish oxidatively transformed carotenoid, or a fractionated component thereof, in an amount sufficient to treat the infection. In particular embodiments the infection is by a bacterium, virus, fungus, protozoan, parasite, or any other infectious agent or condition described herein. Infections which can be treated include, without limitation, a bacterial infection by bacillus, edwardsiella, renibacterium, flavobacterium, aeromonas, mycobacterium, haemophilus, nocardia, pasteurella, pseudomonas, streptococcus, yersinia, or vibrio spp.; a protozoan infection by amoeba, coccidia (i.e., eimeria, haplozoa), ichthyoptheria, gregarina, or microspora spp.; or a viral infection selected from white spot, infectious pancreatic necrosis, viral hemorrhagic septicemia, infectious hematopoetic necrosis, and spring viremia.

The invention features a method of treating a shellfish having, or at risk of, an inflammatory condition by administering to the shellfish a composition including oxidatively transformed carotenoid, or a fractionated component thereof, in an amount sufficient to treat the inflammatory condition.

In certain embodiments the shellfish is a crustacean selected from shrimp, prawn, lobster, crayfish, and crabs. In still other embodiments the shellfish is a mollusk selected from clams, mussels, oysters, winkles, scallops, and squid.

The invention further features a method of treating a crustacean having, or at risk of, shell disease by administering to the crustacean oxidatively transformed carotenoid, or a fractionated component thereof, in an amount sufficient to treat the shell disease.

In any of the above methods, the route of administration can include oral administration of a feed including oxidatively transformed carotenoid, or a fractionated component thereof.

In certain embodiments of any of the above methods, the route of administration includes parenteral administration of a bath including oxidatively transformed carotenoid, or a fractionated component thereof. The parenteral administration can include administration across a gill of the aquatic animal being treated.

In certain embodiments of any of the above methods, the composition includes fractionated oxidatively transformed carotenoid. In certain other embodiments of any of the above methods, the composition includes unfractionated oxidatively transformed carotenoid.

The invention features a method of administering oxidatively transformed carotenoid, or a fractionated component thereof, to an aquatic animal, the method including suspending the oxidatively transformed carotenoid, or a fractionated component thereof, in an aqueous solution and immersing the aquatic animal in the aqueous solution. The oxidatively transformed carotenoid, or a fractionated component thereof, can be formulated as a microemulsion, a microparticle, a solid lipid nanoparticle, or any other formulation described herein. In certain embodiments, the oxidatively transformed carotenoid, or a fractionated component thereof, is formulated as a suspension sized for administration across the gill of the aquatic animal.

In particular embodiments, the aquatic animal is immersed in a bath, dip, flush, or indefinite bath. For example, the aqueous solution in which the aquatic animal is immersed can include from 0.00001% to 0.05% (w/w) oxidatively transformed carotenoid, or a fractionated component thereof (e.g., from 0.1 to 5 ppm, 0.1 to 10 ppm, 0.1 to 50 ppm, 0.5 to 5 ppm, 0.5 to 10 ppm, 0.5 to 50 ppm, 1 to 10 ppm, 1 to 30 ppm, or from 10 to 100 ppm (w/w) oxidatively transformed carotenoid, or a fractionated component thereof).

The step of immersing can be performed from 24 hours preceding to 24 hours following subjecting the aquatic animal to a physical stress (e.g., the physical stress arises from overcrowding; from moving, netting, crowding, cleaning, counting, tagging, or transporting the aquatic animal; from an environmental change, such as changes in salinity, temperature, pH, oxidative stress, or exposure to a chemotherapeutic agent).

The step of immersing can be performed in combination with the oral administration of a feed including oxidatively transformed carotenoid, or a fractionated component thereof.

In certain embodiments, the immersing is performed to treat an infection in the aquatic animal. Infections which can be treated include, without limitation, a bacterial infection by bacillus, edwardsiella, renibacterium, flavobacterium, aeromonas, mycobacterium, haemophilus, nocardia, pasteurella, pseudomonas, streptococcus, yersinia, or vibrio spp.; a protozoan infection by amoeba, coccidia (i.e., eimeria, haplozoa), ichthyoptheria, gregarina, or microspora spp.; a viral infection selected from white spot, infectious pancreatic necrosis, infectious salmon anemia, viral hemorrhagic septicemia, infectious hematopoetic necrosis, and spring viremia; or any infection described herein.

In still other embodiments, the immersing is performed to treat an inflammatory condition in the aquatic animal.

In particular embodiments, the immersion bath can further include an anesthetic (e.g., eugenol or tricaine methanesulfonate), antibiotic (e.g., oxytetracycline, florfenicol, amikacin, ceftazidime, enrofloxacin, nitrofurazone, or trimethoprim sulfadiazine), or parasiticide (e.g., diflubenzuron, fenbendazole, formaldehyde, levamisole phosphate, metronidazole, praziquantel, or trichlorfon).

The aquatic animal can be a fish, such as a catfish, carp, trout, salmon, char, whitefish, sturgeon, tench, roach, pike, pike-perch, sole, turbot, yellowtail, bass, milkfish, tilapia, walleye, gray mullet, eels, angel fish, barb, catfish, cichlids, corydoras, danio, discus, gourami, guppy, koi, roach, minnow, molly, platy, Plecostumas, rainbow and platy variatus, rasbora, shark, sword, tetra, botia, knife fish, lionfish, brackish-archer fish, flounder, golby, half beak, mono, needle fish, pipe fish, puffer, scat, and bumble bee, twin spot damsel, yellowtail damsel, barbed squirrel, wrasse, black-spotted puffer, trigger fish, puffer, butterfly fish, or any other fish described herein.

The aquatic animal can be a shellfish, such as a crustacean (e.g., a shrimp, prawn, lobster, crayfish, crab, or any other crustacean described herein) or a mollusk (e.g., a clam, mussel, oyster, winkle, scallop, squid, or any other mollusk described herein).

The invention features a mollusk feed including from 0.00001% to 0.005% (w/w) oxidatively transformed carotenoid, or a fractionated component thereof (e.g., from 0.1 to 5 ppm, 0.1 to 10 ppm, 0.1 to 50 ppm, 0.5 to 5 ppm, 0.5 to 10 ppm, 0.5 to 50 ppm, 1 to 10 ppm, 1 to 30 ppm, or from 10 to 100 ppm (w/w) oxidatively transformed carotenoid, or a fractionated component thereof).

The invention features a crustacean feed including from 0.00001% to 0.005% (w/w) oxidatively transformed carotenoid, or a fractionated component thereof (e.g., from 0.1 to 5 ppm, 0.1 to 10 ppm, 0.1 to 50 ppm, 0.5 to 5 ppm, 0.5 to 10 ppm, 0.5 to 50 ppm, 1 to 10 ppm, 1 to 30 ppm, or from 10 to 100 ppm (w/w) oxidatively transformed carotenoid, or a fractionated component thereof). The crustacean feed can include from 10% to 60% (w/w) shellfish meal.

In certain embodiments, the crustacean feed is in the form of a crumble for use as a starter feed, or in the form of a pellet or flake for use as a grower feed or a finisher feed.

The invention features a composition including a mixture of oxidatively transformed carotenoid, or a fractionated component thereof, the composition having an effective particle size of from 20 nm to 10 μm (e.g., an effective particle size of from 1 μm to 10 μm, from 20 nm to 1 μm, or from 50 nm to 700 nm). The particulate oxidatively transformed carotenoid can be any particulate formulation described herein. In certain embodiments, the oxidatively transformed carotenoid, or a fractionated component thereof, is encapsulated in an encasing matrix, such as a protein or a carbohydrate.

The invention features a kit, including (i) a particulate composition of the invention; and (ii) instructions for (a) mixing the composition with an aqueous solution and (b) immersing an aquatic animal in the aqueous solution. The kit can further include instructions for immersing the aquatic animal in a bath, dip, flush, or indefinite bath.

The invention further features a kit, including (i) a feed including from 0.00001% to 0.005% (w/w) (e.g., from 0.1 to 5 ppm, 0.1 to 10 ppm, 0.1 to 50 ppm, 0.5 to 5 ppm, 0.5 to 10 ppm, 0.5 to 50 ppm, 1 to 10 ppm, 1 to 30 ppm, or from 10 to 100 ppm (w/w) oxidatively transformed carotenoid, or a fractionated component thereof) oxidatively transformed carotenoid, or a fractionated component thereof; and (ii) instructions for administering the feed to a mollusk.

The invention also features a kit, including (i) a feed including from 0.00001% to 0.005% (w/w) (e.g., from 0.1 to 5 ppm, 0.1 to 10 ppm, 0.1 to 50 ppm, 0.5 to 5 ppm, 0.5 to 10 ppm, 0.5 to 50 ppm, 1 to 10 ppm, 1 to 30 ppm, or from 10 to 100 ppm (w/w) oxidatively transformed carotenoid, or a fractionated component thereof) oxidatively transformed carotenoid, or a fractionated component thereof; and (ii) instructions for administering the feed to a crustacean.

In any of the above methods, compositions, and kits the oxidatively transformed carotenoid, or a component thereof; can be administered orally, intravenously, intramuscularly, ocularly, topically, subcutaneously, intranasally, or by any other route of administration described herein. Desirably, the oxidatively transformed carotenoid, or a component thereof, is incorporated into a feed and administered orally or is dispersed in an immersion bath and administered parenterally (e.g., across a membrane, such as a gill, mouth, gut, eye, or skin of the aquatic animal).

As used herein, the term “ameliorating an effect of physical stress” refers to preventing, eliminating, reducing the severity of, or reducing the recovery time for one or more signs of distress in aquatic species treated using a method of the invention in comparison to untreated aquatic species kept under the same conditions. An effect of physical stress can include an increase in mortality, a reduction in growth rate, an increase in aggression, and/or a decrease in meat quality, among other signs of physical distress known in the art for any given aquatic species, such as shell disease in crustaceans. For example, in tropical fish and goldfish signs of physical distress include clamped fins (fins clamped close against the fish's body); shimmy (the fish moves as if it is swimming fast, but stays in one place); red or white sores (typically caused by fights with other fish or scraping on sharp rocks); fish gasping at the surface; fish crashed at the bottom (a sign of exhaustion); glancing (a behavior where a fish rubs itself on a surface); and loss of appetite. In shrimp signs of physical distress include active swimming by shrimp at the water surface during day-light hours (often caused by low oxygen and/or high temperature); prawn gobics swimming in stress and/or concentrated on the sides of the aquaculture pool; shrimp with black gills; shrimp with white discoloration on their tails; shrimp with papery shells and a body that pushes in easily; shrimp with black spots; and shrimp with fuzzy growth on outside shell (an indication that growth is slow and the shrimp are not molting).

By an “amount sufficient” is meant the amount of oxidatively transformed carotenoid, or a fractionated component thereof, required to treat or prevent inflammation, infection, and/or physical stress or a disease associated with inflammation, infection, and/or physical stress. The effective amount of a pharmaceutical composition of the invention used to practice the invention for therapeutic or prophylactic treatment of conditions resulting in or contributed to inflammation, infection, and/or physical stress varies depending upon the manner of administration, the age, body weight, and general health of the aquatic species. Ultimately, the attending veterinarian will decide the appropriate amount and dosage regimen. Such amount is referred to as an “amount sufficient.”

As used herein, the term “bath” refers to a treatment in which oxidatively transformed carotenoid, or a fraction thereof, is suspended in water in which an aquatic animal is immersed for at least 15 minutes and less than 24 hours.

As used herein, “carotenoid” refers to naturally-occurring pigments of the terpenoid group that can be found in plants, algae, bacteria, and certain animals, such as birds and shellfish. Carotenoids include carotenes, which are hydrocarbons (i.e., without oxygen), and their oxygenated derivatives (i.e., xanthophylls). Examples of carotenoids include lycopene; β-carotene; zeaxanthin; echinenone; isozeaxanthin; astaxanthin; canthaxanthin; lutein; citranaxanthin; β-apo-8′-carotenic acid ethyl ester; hydroxy carotenoids, such as alloxanthin, apocarotenol, astacene, astaxanthin, capsanthin, capsorubin, carotenediols, carotenetriols, carotenols, cryptoxanthin, decaprenoxanthin, epilutcin, fucoxanthin, hydroxycarotenones, hydroxyechinenones, hydroxylycopene, lutein, lycoxanthin, neurosporine, phytoene, phytofluoene, rhodopin, spheroidene, torulene, violaxanthin, and zeaxanthin; and carboxylic carotenoids, such as apocarotenoic acid, β-apo-8′-carotenoic acid, azafrin, bixin, carboxylcarotenes, crocetin, diapocarotenoic acid, neurosporaxanthin, norbixin, and lycopenoic acid.

As used herein, the term “dip” refers to a treatment in which oxidatively transformed carotenoid, or a fraction thereof, is suspended in water in which an aquatic animal is immersed for at least 1 second and less than 15 minutes.

As used herein, the terms “effective particle size” and “particle size” are used interchangeably and refer to a mixture of particles having a distribution in which 50% of the particles are below and 50% of the particles are above a defined measurement. The “effective particle size” refers to the volume-weighted median diameter as measured by a laser/light scattering method or equivalent, wherein 50% of the particles, by volume, have a smaller diameter, while 50% by volume have a larger diameter. The effective particle size can be measured by conventional particle size measuring techniques well known to those skilled in the art. Such techniques include, for example, sedimentation field flow fractionation, photon correlation spectroscopy, light scattering (e.g., with a Microtrac UPA 150), laser diffraction, and disc centrifugation.

By “fish” or “finfish” is meant any aquatic vertebrate animal that is covered with scales and bears fins. Fish include any of the considerable variety of fresh, brackish, or salt water fish species including, without limitation, catfish, carp, trout, salmon, char, whitefish, sturgeon, tench, roach, pike, pike-perch, sole, turbot, yellowtail, bass, milkfish, tilapia, walleye, gray mullet, eels and tropical fish (e.g., fresh, brackish, and salt water tropical fish, such as angel fish, barb, catfish, cichlids, corydoras, danio, discus, eel, gourami, guppy, koi, loach, minnow, molly, platy, plecostumas, rainbow and platy variatus, rasbora, shark, sword, tetra, botia, knife fish, and lionfish, brackish-archer fish, flounder, golby, half beak, mono, needle fish, pipe fish, puffer, scat (green and red), bumble bee, twin spot damsel, yellowtail damsel, barbed squirrel, wrasse, black-spotted puffer, trigger fish, puffer, and butterfly fish.

As used herein, the term “flush” refers to a treatment in which oxidatively transformed carotenoid, or a fraction thereof, is suspended in water which is added to the inflow area of a vessel (e.g., a raceway or narrow vat) and the aquatic animal is immersed in the solution containing the oxidatively transformed carotenoid, or a fraction thereof, as it passes over them with the water current. This is similar to a dip procedure except that the aquatic animal need not be removed from their normal holding area.

As used herein “fractionated” refers to a composition containing the oligomeric material formed in the production of the oxidatively transformed carotenoid mixture. Methods of fractionating oxidatively transformed carotenoid mixtures into components are described in U.S. Pat. No. 5,475,006 and U.S. Ser. No. 08/527,039, each of which are incorporated herein by reference.

As used herein, the term “indefinite bath” refers to a treatment in which oxidatively transformed carotenoid, or a fraction thereof, is suspended in water in which an aquatic animal is immersed for at least 24 hours. For example, the oxidatively transformed carotenoid, or a fraction thereof, can be suspended the water of an aquarium without need for further water change or immediate retreatment.

By “infection” is meant the invasion of an aquatic animal by a microbe, e.g., a bacterium, fungus, protozoan, parasite, or virus. The infection may include, for example, the excessive multiplication of microbes that are normally present in or on the body of an aquatic animal or multiplication of microbes that are not normally present in or on an aquatic animal. An aquatic animal is suffering from a microbial infection when an excessive amount of a microbial population is present in or on the animal's body or when the presence of a microbial population(s) is damaging the cells or causing pathological symptoms to a tissue of the aquatic animal.

By “oxidative stress” is meant the stress caused by a pathological overproduction of reactive oxygen species in tissues. Oxidative stress is one general mechanism of toxicity associated with exposure to xenobiotics (e.g., oxidative stress can be induced by exposure to anthropogenic contaminants, such as persistent organic pollutants, heavy metals, and bleach, and induced by toxins produced by blooms of cyanobacteria).

As used herein “oxidatively transformed carotenoid” refers to a carotenoid which has been reacted with up to 6 to 8 molar equivalents of oxygen, or an equivalent amount of oxygen from another oxidizing agent, resulting in a mixture of very low molecular weight oxidative cleavage products and a large proportion of oligomeric material (i.e., that component of the oxidatively transformed carotenoid having a median molecular weight of about 900 Daltons or more and an upper limit of 8,000 Daltons, 10,000 Daltons, 12,000 Daltons, or more). The resulting reaction produces a mixture that includes molecular species having molecular weights ranging from about 100 to about 8,000 Daltons, 10,000 Daltons, 12,000 Daltons, or more. The oligomeric material is believed to be formed by the many possible chemical recombinations of the various oxidative fragments that are formed. Methods of making oxidatively transformed carotenoid are described in U.S. Pat. No. 5,475,006 and U.S. Ser. No. 08/527,039, each of which are incorporated herein by reference. As used herein, the term “OxBC” refers specifically to oxidatively transformed carotenoid derived from β-carotene.

As used herein, the term “shell disease” refers to a condition characterized by the progressive degradation of exoskeletal chitin accompanied by melanization of the affected region and includes both the endemic and epizootic forms of the condition. Otherwise known as black spot, rust spot or burned spot, shell disease is reported to affect numerous crustacean species worldwide. It is believed that shell disease occurs when the process of chitin deposition fails to keep pace with the normal processes of the surficial microbial community, and lesions follow. The most severe cases are found on crustaceans that molt less often, such as primarily ovigerous female lobsters, and found where crustaceans are raised in poor quality environments (e.g., exposed to extreme temperatures, pollution, hypoxia, and/or excess organic matter).

As used herein “shellfish” refers to aquatic invertebrates, including mollusks and crustaceans. Mollusks include, without limitation, clams, mussels, oysters, winkles, scallops, and squid. Crustaceans include, without limitation, shrimp, prawn, lobster, crayfish, and crabs.

As used herein, the term “shellfish meal” refers to a component of a feed made from ground shellfish.

As used herein, the term “treating” refers to administering a pharmaceutical composition for prophylactic and/or therapeutic purposes. To “prevent disease” refers to prophylactic treatment of an aquatic animal that is not yet ill, but who is susceptible to, or otherwise at risk of; a particular disease. Prophylactic use can be used to treat an aquatic animal not yet ill to reduce the likelihood of disease, reduce the severity of disease, or to ameliorate one or more symptoms of a disease. To “treat disease” or use for “therapeutic treatment” refers to administering treatment to an aquatic animal already suffering from a disease to improve or stabilize the aquatic animal's condition. Thus, in the claims and embodiments, treating is the administration to an aquatic animal either for therapeutic or prophylactic purposes. As used herein, “at risk of” refers to aquatic animals prone to inflammation, infection, and/or physical stress. Aquatic animals can be prone to inflammation, infection, and/or physical stress, for example, by virtue of (i) having an autoimmune condition, (ii) exposure to allergens, (iii) exposure to infectious microbes, or (iv) exposure to poor environments (e.g., overcrowding, pollution, extreme temperatures, chemotherapeutic agents, and/or extreme pH).

The compositions and methods of the invention can be used to treat an inflammation, infection, and/or physical stress in an aquatic animal. The inflammation, infection, and/or physical stress can be any inflammation, infection, and/or physical stress described herein.

Other features and advantages of the invention will be apparent from the following Detailed Description and the Claims.

### BRIEF DESCRIPTION OF THE DRAWINGS

FIG. 1 is a graph showing the effect of OxBC on respiratory burst activity of naïve primary trout leukocyte cultures. Leukocytes (10⁶ cells/well) were incubated for 1 hour in the presence of the indicated concentrations of OxBC or control compounds. Leukocyte respiratory burst activity was assessed using a standard nitroblue tetrazolium (NBT) reduction assay. Lipopolysaccharide isolated from Aeromonas salmonicida (A sal LPS) and phorbol myristate acetate (PMA) were used as reference controls. Results indicate a significant increase in respiratory burst activity for leukocytes treated with 10 μM OxBC (* p<0.05 Student's t-test versus DMSO control).

FIG. 2 is a graph showing the effect of OxBC on bactericidal activity of primary trout leukocyte cultures. Trout leukocytes were primed for 24 hours by incubation with the indicated concentration of OxBC (1.0 μM, 10 μM, or 50 μM) and were then allowed to recover for 24 hours in fresh media containing no OxBC. Following the recovery period, leukocytes were subjected to a pathogen challenge by the addition of fresh cultures of Aeromonas salmonicida (ATCC 33658) to the media. Leukocyte bactericidal activity was evaluated at 0 (bars on the left for control and OxBC concentrations) and 3 hours (bars on the right for control and OxBC concentrations) post-challenge by measuring the level of viable bacteria by using a standard MTT assay. A reduction in the level of viable bacteria indicates an increase in leukocyte bactericidal activity. The first series of bars show the effects of OxBC on trout primary leukocyte bactericidal activity, and the second series of bars show the results of control experiments conducted to confirm that OxBC has no direct effect on bacterial viability. Results from the 3-hour post challenge group suggest the presence of a dose dependant response to OxBC with a significant increase in bactericidal activity observed for leukocytes primed with 50 μM OxBC relative to non-primed controls (Student's t-test, **p<0.001). Control experiments with bacteria cultured in the presence of OxBC indicated no direct cytotoxic effect of the compound on A. salmonicida.

### DETAILED DESCRIPTION

The invention provides compositions, methods, and kits for the administration of oxidatively transformed carotenoid and fractionated components thereof. The compositions, methods, and kits can be useful in aquaculture, such as in open system farming, closed system farming, sea ranching, and raising ornamental fish. The compositions, methods, and kits can also be useful for the maintenance of normal immune function in finfish and shellfish.

Open system farming is the term used to describe the process of aquaculture farming in cages or pens that are open to the sea. These typically include nets suspended from either a floating metal framework or from round plastic floating structures, which may or may not be anchored to the seabed. Being open to the surrounding water means that the fish can potentially be infected with and/or transmit water borne disease and sea lice infestations to/from wild stocks. For example, juvenile salmon are grown in hatcheries from eggs produced by broodstock, when the eggs hatch the young fish are called alevins until they discard their yolk sac when they are termed fry. Fry are grown in freshwater tanks, where they undergo a series of size sortings called gradings, a process to ensure that fish of a similar size are kept together. They remain in these tanks until they undergo a process called smoltification, a physiological process that enables the fish to live in seawater, which normally occurs when they are 12-18 months old. After smoltification the fish are termed smolts. Once transferred to sea cages the salmon take between 18 months and 2 years to reach harvestable size of 3-4 kg.

Closed system farming is the term used to describe aquaculture farming in enclosed systems not open to any water body. There are a number of methods that can be used, including enclosed ponds, tanks, or raceways. Closed system farming has been used to raise shrimp in ponds and ornamental fish in tanks.

Sea ranching is the process of releasing artificially raised juvenile aquatic animals into the sea, allowing them to mature to market size and then recapturing them. For example, tuna ranching involves catching juvenile tuna from the wild and growing them further in cages. Typically, juvenile fish, that have not had a chance to reproduce, are caught in nets and towed slowly through the sea to near shore waters where they are transferred to net-pens or cages for on-growing. After several months, when the tuna have reached optimal market size, they are harvested and sold primarily for the sushi and sashimi markets.

Other finfish species currently farmed include rainbow trout, sea trout, Atlantic halibut, Atlantic cod, and turbot. The trout species are farmed in closed systems such as raceways, while the halibut and turbot are raised in enclosed tanks containing seawater.

Diseases can occur amongst farmed fish and crustaceans due to the physical stressors associated with an aquaculture environment and because diseases and parasites are more easily transferred between individuals due to their close proximity.

### Administration

The invention features compositions, kits, and methods for treating inflammation, infection, and/or physical stress in an aquatic animal. For oxidatively transformed carotenoid, typical dose ranges are from about 5 μg/kg to about 50 mg/kg of body weight per day. Desirably, a dose of between 5 μg/kg and 5 mg/kg of body weight, or 5 μg/kg and 0.5 mg/kg of body weight, is administered. For a component of oxidatively transformed carotenoid, typical dose ranges are from about 0.05 μg/kg to about 500 μg/kg of body weight per day. Desirably, a dose of between 0.05 μg/kg and 50 μg/kg of body weight, or 0.05 μg/kg and 5 μg/kg of body weight, is administered. The dosage of oxidatively transformed carotenoid, or a fractionated component thereof, to be administered is likely to depend on such variables as the species, diet, and age of the aquatic animal. Standard trials may be used to optimize the dose and dosing frequency of the oxidatively transformed carotenoid or a fractionated component thereof. The oxidatively transformed carotenoid can be administered as part of the diet of the aquatic animal and/or as part of a immersion treatment administered to the aquatic animal.

One optional mode of treatment is to treat most or all stages of growth, from the brood stock, through egg spawning and fertilization, and then through juvenile stages.

For marine shrimp species, the life stages comprise fertilized eggs, larvae (developmental stages include nauplier, protozoel, and mysis), postlarval, juveniles, sub-adults, spawning adults, and non-spawning adults.

Brood stocks of marine organisms may be treated with oxidatively transformed carotenoid prior to spawning. Conditioned brood stock are washed and prepared for spawning according to normal hatchery operations procedures. The brood stock can be bathed in a suspension of oxidatively transformed carotenoid at concentration of between 0.00001% to 0.05% (w/w) (e.g., from 0.1 to 5 ppm, 0.1 to 10 ppm, 0.1 to 50 ppm, 0.5 to 5 ppm, 0.5 to 10 ppm, 0.5 to 50 ppm, 1 to 10 ppm, 1 to 30 ppm, or from 10 to 100 ppm by weight) oxidatively transformed carotenoid. This bath treatment is initiated when the marine organisms are placed in spawning trays with the intent that the brood stock will pump the oxidatively transformed carotenoid-containing water through their water tubular system and provide a treatment of the ova and sperm with the oxidatively transformed carotenoid. This treatment may be combined with other treatments.

In aquatic animals various growth stages may be treated with oxidatively transformed carotenoid during the processes of screening and grading. Screening is a process in which early life stage aquatic animals in a tank are split into several tanks to prevent crowding, or alternatively, when the tanks are cleaned. Grading is a part of the screening process in which the aquatic animals are screened through progressive mesh sizes of screens to separate them by size. Larvae from static or continuous culture tanks can be treated when concentrated by screening for tank cleaning or grading. In static culture tanks, this typically occurs every two to three days but in continuous culture tanks, draining and grading occurs less frequently (3 to 6 day intervals). The concentrated larvae are resuspended in a minimal volume of sterile seawater (SSW) containing oxidatively transformed carotenoid at a concentration of from of between 0.00001% to 0.05% (w/w) (e.g., from 0.1 to 5 ppm, 0.1 to 10 ppm, 0.1 to 50 ppm, 0.5 to 5 ppm, 0.5 to 10 ppm, 0.5 to 50 ppm, 1 to 10 ppm, 1 to 30 ppm, or from 10 to 100 ppm by weight) oxidatively transformed carotenoid for 10 minutes to 1 hour. Untreated control larvae from separate tanks can be used and quantitatively characterized (survival, growth, time to metamorphosis) in parallel with the treatment group.

Addition of oxidatively transformed carotenoid to selected feeds for feeding both larvae and juveniles can be employed, either in alternative to, or in addition to, the immersion treatments described herein.

Fish in both commercial and home aquariums may be treated with oxidatively transformed carotenoid. Due to their confinement and controlled environment, it is possible to treat fish at all life stages. The tanks may be treated with a concentration of from between 0.00001% to 0.05% (w/w) (e.g., from 0.1 to 5 ppm, 0.1 to 10 ppm, 0.1 to 50 ppm, 0.5 to 5 ppm, 0.5 to 10 ppm, 0.5 to 50 ppm, 1 to 10 ppm, 1 to 30 ppm, or from 10 to 100 ppm by weight) oxidatively transformed carotenoid.

### Therapy

Therapy according to the invention may be performed to treat or prevent inflammation, infection, and/or physical stress, or a disease associated with inflammation, infection, and/or physical stress, in an aquatic animal.

Inflammation is a protective reaction of the host in response to injury, resulting in specific morphological and chemical changes in tissues and cells. In fishes as well, much basic research has been conducted on the process of inflammatory leukocyte migration, which is the most characteristic event of the acute phase. The first response of a host to injury is vasodilatation, followed by increased vascular permeability. These vascular reactions have significance in understanding the mechanism of leukocyte migration, which occurs through the injured blood vessels and in response to chemical mediators converted from certain plasma proteins. Neutrophils migrate more quickly than do monocytes and macrophages during acute inflammation, as has been observed in many fish species. These leukocytes are phagocytes which act to remove irritants, bacteria, or damaged cells and tissues. Leukocytic infiltration in inflammation can be explained by chemical mediators. Complement factors, leucotriene B₄ and a lymphokine, have been identified as chemotactic and chemokinetic factors for fish neutrophils. Besides these host factors, bacterial formyl peptides are reported to be chemoattractive for plaice (flat fish) neutrophils. The process of leukocytic migration in various types of inflammation has specific features, which can be modulated using the methods, kits, and compositions of the invention.

Raising shellfish and finfish in an aquaculture hatchery or aquarium setting subjects an aquatic animal to numerous physical stressors on a daily basis. These physical stressors can have a detrimental impact on their health. Such physical stressors include the environmental disturbances caused by normal hatchery operations, such as moving, netting, pumping, crowding, cleaning, water-changing, sampling, counting, tagging, fin-clipping, transporting, and stocking the aquatic animal. Physical stressors also include poor environment associated changes in salinity, temperature, pH, oxidative stress, or exposure to a chemotherapeutic agent. The effects of physical stress can include an increase in mortality, a reduction in growth rate, an increase in aggression, and/or a decrease in meat quality, among other signs of physical distress known in the art for any given aquatic species. Specific examples of the symptoms of physical stress include, without limitation, shell disease in crustaceans; clamped fins, shimmy, red or white sores, fish gasping at the surface, fish crashed at the bottom, glancing, and loss of appetite in finfish; and active swimming at the water surface during day-light hours, prawn gobies swimming in stress and/or concentrated on the sides of the aquaculture pool, black gills, white discoloration of the tails, papery shells, and black spots in shrimp. The methods, kits, and compositions of the invention can be used to treat physical stress in an aquatic animal.

The misuse of antibiotics to control infections in aquaculture has resulted in the development of resistant strains, which have rendered antibiotic treatments ineffective. Moreover, the horizontal transfer of resistance determinants to human pathogens and the presence of antibiotic residues in aquaculture products for human consumption constitute important threats to public health. The methods, kits, and compositions of the invention can be used to treat an infection in an aquatic animal by modulating the immune system of the aquatic animal. The methods, kits, and compositions of the invention can be used to treat a variety of infectious diseases and pathogens including, without limitation, viral (e.g., infectious hypodermal and haematopoietic necrosis, white spot syndrome, baculovirus penaei disease, hepatopancreas parvovirus disease, taura syndrome, yellow head disease, infectious salmon anemia, infectious pancreatic necrosis, viral hemorrhagic septicemia, and spring viremia); bacterial (e.g., infections by bacillus, edwardsiella, renibacterium, flavobacterium, aeromonas, mycobacterium, haemophilus, nocardia, pasteurella, pseudomonas, streptococcus, yersinia, or vibrio spp., rickettsial infections, and chlamydial infections); protozoan (e.g., infections by amoeba, coccidia (eimeria, haplozoa), ichthyoptheria, gregarina, microspora spp., and ciliate disease); fungal diseases (e.g., infections by lagenidium, fusarium, or haliphthoros spp.), among other parasites (e.g., paramoebiasis of lobsters). The aquatic animal being treated can be any finfish or shellfish described herein.

### Combination Therapy

Therapy according to the invention may be performed alone or in conjunction with another therapy. For example, the aquatic animal can be treated with an aqueous solution including oxidatively transformed carotenoid and a second agent selected from an anesthetic (e.g., eugenol or tricaine methanesulfonate), antibiotic (e.g., oxytetracycline, florfenicol, amikacin, ceftazidime, enrofloxacin, nitrofurazone, or trimethoprim sulfadiazine), or parasiticide (e.g., diflubenzuron, fenbendazole, formaldehyde, levamisole phosphate, metronidazole, praziquantel, or trichlorfon).

### Aquaculture Feeds

Therapy according to the invention can include treating an aquatic animal with an aquaculture feed containing from 0.00001% to 0.05% (w/w) oxidatively transformed carotenoid, or a fractionated component thereof. The feed can be a fish feed or a shellfish feed, such as a crustacean feed. The feed can be prepared using methods known in the art, including those described below.

Fish Feeds

Different species of farmed finfish require different diets. Some species such as tilapia and catfish can be fed on an entirely vegetarian diet, while the majority of farmed species are fed a carnivorous diet. The feed for carnivorous fish includes fishmeal and fish oil derived from wild caught species of small pelagic fish predominantly anchovy, jack mackerel, blue whiting, capelin, sandeel and Medhaden. These pelagic fish are processed into fishmeal and fish oil, with the final product being a pelleted (for larger fish) or flaked (for juveniles) feed. The other components of the feed pellet are vegetable protein, vitamins, minerals and pigment as required.

It is preferable that fish be fed oxidatively transformed carotenoid as a mixture with pre-formed fish food pellets, crumbles, or other fish food forms, e.g., commercially available fish foods, or as an ingredient in a fish food including other well-known ingredients included in commercial fish food formulations so as to provide a nutritionally balanced complete fish food, including, but not limited to: vegetable matter, e.g., flour, meal, starch or cracked grain produced from a crop vegetable such as wheat, alfalfa, corn, oats, potato, rice, and soybeans; cellulose in a form that may be obtained from wood pulp, grasses, plant leaves, and waste vegetable matter such as rice or soy bean hulls, or corn cobs; animal matter, e.g., fish and shellfish (e.g., shrimp or crab) meal, oil, protein or solubles and extracts, krill, meat meal, bone meal, feather meal, blood meal, or cracklings; algal matter; yeast; bacteria; vitamins, minerals, and amino acids; organic binders or adhesives; and chelating agents and preservatives. A wide variety of commercial fish foods may be used in combination with oxidatively transformed carotenoid including, without limitation, “BioDiet BROOD” pellets (Bioproducts, Inc., Warrenton, Oreg.) or “40% Bass Food (Star Milling Co., Perris, Calif.), and “Catfish Food” (Star Milling Co., Perris, Calif.).

For feeding smaller fish species, such as many varieties kept in home aquariums, it may be necessary to provide a fish food having a correspondingly smaller size, as will be readily appreciated by the skilled artisan. For example, small tropical fish species can be fed fish food, derived from commercial fish food pellets with added oxidatively transformed carotenoid, that has been passed through a blender (set on chop) to reduce the size of the food particles to an acceptable size.

Crustacean Feeds

Typically, a number of different feed types are used in the production of a crustacean crop. There exist three major feed categories, broadly referred to as starter, grower, and finisher. The major differences between these feeds are the size of the feed. The crustacean feed is produced in two general forms, steam pelleted and extruded, loosely discriminated by their intended culture species. Microencapsulation and suspension type feeds, (that are a homogenization of ingredients, which is added to the larval rearing water) are feeds that are designed for the larval rearing stage of crustacean species. Flaked feeds are also used. The starter type feeds are either in the form of a small pellet or a “crumble” which has been produced by passing already prepared pellets through machinery that physically cracks the pellets into smaller particles. The “crumbled” feed is then graded into different size fractions for different size organisms. The grower and finisher type feeds are in pellet form only. Again the major difference is size of the pellets, with the finisher pellets being somewhat larger than the grower pellets.

A number of biological and non-biological materials are used to manufacture crustacean feeds, which typically include one or more sources of shellfish meal (e.g., krill meal, crab meal, or langostine meal) in addition to vegetable matter, animal matter, and oil.

Feed Manufacture

The manufacture of aquaculture feeds is the process of combining ingredients to form a mixture designed to provide a variety of nutrients and non-nutrient compounds in a practical form to farmed aquatic animals. Non nutrient components include fillers such as limestone and pellet binders that offer no nutritional contribution to the integrated components. Feeds can be designed to meet a number of goals, including rapid growth for market, cost effective growth parameters, successful reproduction or low pollution.

Steam pelleting through the process of compression produces dense pellets that sink in the water. This process involves the use of moisture, heat and pressure to bind together the small ground particles into pellets. Steam is added to the ground mixture to partially cook the starches found in the ingredients. This steam addition also adds an amount of water that assists in the cooking process. First, the ingredients are transferred to the grinding system for size reduction. After this stage the ground meal is transferred into the conditioning system. This is the point for the addition of water and steam. The conditioning system is a sealed system where the ground ingredients are agitated with radial mixers under the influence of moisture and temperature supplied by steam at varying pressure. The conditioned meal is introduced into the pellet mills where the hot moist mash is compressed through the pellet dies. These pellet dies shape the mash into pellets, which are cut off to length at the face of the pellet die. These hot pellets are then transferred to a cooling system where ambient air is blown over the pellets to cool and reduce the moisture level. From this point the cool pellets are transferred to the bulk storage silos as a finished product for packing into bags for storage and final dispatch.

Steam extrusion can be used to manufacture an aquaculture feed. The extrusion method is similar to the process of steam pelleting and involves mixing, grinding, and conditioning of ingredients. However once the conditioned mash of ingredients is about to be pelleted it enters the extrusion barrel where it is subjected to extremely high pressures and temperature for a short period of time.

Cold pressing is however used in the production of certain fish feeds. This process involves the mixing of ingredients with a liquid, usually water until a stiff dough like consistency is achieved. The mix is then formed into pellets by extrusion through a die plate at room temperature.

Agrommalation is a method in which finely ground ingredients are mixed with a water activated binder. This mix is then introduced onto an angled spinning disk and the moist mash is agrommalated into small spheres. The pellets can be either in the “dry” form with moisture of approximately 10% or a “semi-moist” pellet with moisture content of 25%.

Flaking is the process where the feed ingredients are mixed with water to form a slurry mixture. The most common form of production is the introduction as a thin film onto a slowly revolving heated drum. Due to the thickness of the slurry the mixture quickly dries and is scrapped off the drum before the next full rotation and addition of more slurry. The drums are usually steam heated. Other methods include the use of freeze-drying or oven drying.

### Micronized and Microparticulate Formulations

A micronized, microparticulate, or nanoparticulate formulation of oxidatively transformed carotenoid can be used in the methods of the invention to produce the desired suspension in aqueous solution sized for parenteral administration to the aquatic animal. The microparticulate and nanoparticulate formulations allow the oxidatively transformed carotenoid to be readily dispersed, without settling, in an immersion bath, and are sized to enable parenteral administration of the oxidatively transformed carotenoid to an aquatic animal.

In one approach, the oxidatively transformed carotenoid is mixed with a matrix material and milled in order to obtain micron or submicron particles. The milling process can be a dry process, e.g., a dry roller milling process, or a wet process, i.e., wet-grinding. A wet-grinding process is described in U.S. Pat. Nos. 4,540,602, 5,145,684, 6,976,647 and EPO 498,482, the disclosures of which are hereby incorporated by reference. Thus, the wet grinding process can be practiced in conjunction with a liquid dispersion medium and dispersing or wetting agents such as described in these publications. Useful liquid dispersion media include safflower oil, ethanol, n-butanol, hexane, or glycol, among other liquids selected from known biocompatible excipients (see U.S. Pat. Nos. 4,540,602 and 5,145,684), and can be present in an amount of 2.0-70%, 3-50%, or 5-25% by weight based on the total weight of the oxidatively transformed carotenoid in the formulation.

The grinding media for the particle size reduction step can be selected from rigid media, typically spherical in shape, though non-spherical grinding media could also be used. The grinding media preferably can have a mean particle size from 1 mm to about 500 microns. For fine grinding, the grinding media particles can have a mean particle size from about 0.05 to about 0.6 mm. Smaller size grinding media will result in smaller size oxidatively transformed carotenoid particles as compared to the same conditions using larger sized grinding media. In selecting material, grinding media with higher density, e.g., glass (2.6 g/cm³), zirconium silicate (3.7 g/cm³), and zirconium oxide (5.4 g/cm³) and 95% zirconium oxide stabilized with yttrium, can be utilized for more efficient milling. Alternatively, polymeric grinding media can be used. Polymeric resins suitable for use herein are chemically and physically inert, substantially free of metals, solvent and monomers, and of sufficient hardness and friability to enable them to avoid being chipped or crushed during grinding. Suitable polymeric resins include, without limitation, crosslinked polystyrenes, such as polystyrene crosslinked with divinylbcnzene, styrene copolymers, polycarbonates, polyacetals, such as Delrin™, vinyl chloride polymers and copolymers, polyurethanes, polyamides, poly(tetrafluoroethylenes), e.g., Teflon™, and other fluoropolymers, high density polyethylenes, polypropylenes, cellulose ethers and esters such as cellulose acetate, polyhydroxymethacrylate, polyhydroxyethyl acrylate, and silicone containing polymers such as polysiloxanes.

Grinding can take place in any suitable grinding mill. Suitable mills include an airjet mill, a roller mill, a ball mill, an attritor mill, a vibratory mill, a planetary mill, a sand mill and a bead mill. A high energy media mill is preferred when small particles are desired. The mill can contain a rotating shaft.

The preferred proportions of the grinding media, oxidatively transformed carotenoid, the optional liquid dispersion medium, and dispersing, wetting or other particle stabilizing agents present in the grinding vessel can vary within wide limits and depend on, for example, the size and density of the grinding media, the type of mill selected, the time of milling, etc. The process can be carried out in a continuous, batch or semi-batch mode. In high energy media mills, it can be desirable to fill 80-95% of the volume of the grinding chamber with grinding media. On the other hand, in roller mills, it frequently is desirable to leave the grinding vessel up to half filled with air, the remaining volume including the grinding media and the liquid dispersion media, if present. This permits a cascading effect within the vessel on the rollers which permits efficient grinding. However, when foaming is a problem during wet grinding, the vessel can be completely filled with the liquid dispersion medium or an anti-foaming agent may be added to the liquid dispersion.

The attrition time can vary widely and depends primarily upon the mechanical means and residence conditions selected, the initial and desired final particle size, among other factors. For roller mills, processing times from several days to weeks may be required. On the other hand, milling residence times of less than about 2 hours are generally required using high energy media mills. After attrition is completed, the grinding media is separated from the milled oxidatively transformed carotenoid particulate product (in either a dry or liquid dispersion form) using conventional separation techniques, such as by filtration, or sieving through a mesh screen.

To produce oxidatively transformed carotenoid particles having an effective particle size of less than about 1 micron, the grinding media can be made from beads having a size ranging from 0.05 mm to 4 mm. For example, high energy milling of oxidatively transformed carotenoid and carrier matrix with yttrium stabilized zirconium oxide 0.4 mm beads for a milling residence time of 25 minutes to 1.5 hours in recirculation mode at 1200 to 3000 RPM. In another approach, high energy milling of oxidatively transformed carotenoid particles with 0.1 mm zirconium oxide balls for a milling residence time of 2 hours in batch mode can be used. The milling concentration can be from about 10% to about 30% oxidatively transformed carotenoid/matrix carrier by weight in comparison to the milling slurry weight, which can contain a wetting and/or dispersing agent to coat the initial oxidatively transformed carotenoid/carrier matrix suspension so a uniform feed rate may be applied in continuous milling mode. Alternatively, batch milling mode is utilized with a milling media containing an agent to adjust viscosity and/or provide a wetting effect so that the oxidatively transformed carotenoid is well dispersed amongst the grinding media.

Oxidatively transformed carotenoid particles can also be prepared by homogeneous nucleation and precipitation in the presence of a wetting agent or dispersing agent using methods analogous to those described in U.S. Pat. Nos. 5,560,932 and 5,665,331, which are specifically incorporated by reference. Such a method can include the steps of: (1) dispersing oxidatively transformed carotenoid in a suitable liquid media; (2) adding the mixture from step (1) to a mixture including at least one dispersing agent or wetting agent such that at the appropriate temperature, the oxidatively transformed carotenoid is dissolved; and (3) precipitating the formulation from step (2) using an appropriate anti-solvent. The method can be followed by removal of any formed salt, if present, by dialysis or filtration and concentration of the dispersion by conventional means. In one embodiment, the oxidatively transformed carotenoid particles are present in an essentially pure form and dispersed in a suitable liquid dispersion media. In this approach the oxidatively transformed carotenoid particles are a discrete phase within the resulting mixture. Useful dispersing agents, wetting agents, solvents, and anti-solvents can be experimentally determined.

Oxidatively transformed carotenoid particles can also be prepared by high pressure homogenization (see U.S. Pat. No. 5,510,118). In this approach oxidatively transformed carotenoid particles are dispersed in a liquid dispersion medium and subjected to repeated homogenization to reduce the particle size of the oxidatively transformed carotenoid to the desired effective average particle size. The oxidatively transformed carotenoid particles can be reduced in size in the presence of at least one or more dispersing agents or wetting agents. Alternatively, the oxidatively transformed carotenoid particles can be contacted with one or more dispersing agents or wetting agents either before or after attrition. Other materials, such as a diluent, can be added to the oxidatively transformed carotenoid/dispersing agent mixture before, during, or after the size reduction process. For example, unprocessed oxidatively transformed carotenoid can be added to a liquid medium in which it is essentially insoluble to form a premix (i.e., about 0.1-60% w/w oxidatively transformed carotenoid, and about 20-60% w/w dispersing agents or wetting agents). The apparent viscosity of the premix suspension is preferably less than about 1000 centipoise. The premix can then be transferred to a microfluidizer and circulated continuously first at low pressures, and then at maximum capacity (i.e., 3,000 to 30,000 psi) until the desired particle size reduction is achieved.

Foaming during the nanosizing can present formulation issues and can have negative consequences for particle size reduction. For example, high levels of foam or air bubbles in the mill can cause a drastic increase in viscosity rendering the milling process inoperable. Even a very low level of air presence can dramatically reduce milling efficiency causing the desired particle size unachievable. This may be due to the resultant air in the mill cushioning the milling balls and limiting grinding efficiency. The air also can form a microemulsion with the milled ingredients which presents many issues with respect to the delivery of an accurate dose and palatability. Addition of a small amount of simethicone is a very effective anti-foaming agent which minimizes milling variability or special handling techniques to avoid the introduction of air into the milling process.

The oxidatively transformed carotenoid particles can be prepared with the use of one or more wetting and/or dispersing agents, which are, e.g., adsorbed on the surface of the oxidatively transformed carotenoid particle. The oxidatively transformed carotenoid particles can be contacted with wetting and/or dispersing agents either before, during, or after size reduction. Generally, wetting and/or dispersing agents fall into two categories: non-ionic agents and ionic agents. The most common non-ionic agents are excipients which are contained in classes known as binders, fillers, surfactants and wetting agents. Limited examples of non-ionic surface stabilizers are hydroxypropylmethylcellulose, polyvinylpyrrolidone, plasdone, polyvinyl alcohol, pluronics, tweens, and polyethylene glycols (PEGs). Ionic agents are typically organic molecules bearing an ionic bond such that the molecule is charged in the formulation, such as long chain sulfonic acid salts (e.g., sodium lauryl sulfate and dioctyl sodium sulfosuccinate).

Excipients, such as wetting and dispersing agents, can be applied to the surface of the oxidatively transformed carotenoid nanoparticulate via spray drying, spray granulation, or spray layering process. These procedures are well known in those skilled in the art. It is also common to add additional excipients prior to removal of solvent in the nanoparticulate suspension to aid in the dispersion of the solid composition in medium in which the solid composition will be exposed (e.g. saliva) to further prevent agglomeration and/or particle size growth of the small oxidatively transformed carotenoid particles. An example of such an additional excipient is a redispersing agent. Suitable redispersing agents include, without limitation, sugars, polyethylene glycols, urea and quarternary ammonium salts.

Oxidatively transformed carotenoid particles can also be prepared as solid lipid nanoparticles or as spray dried emulsions. Techniques for making solid lipid nanoparticles and spray dried emulsions incorporating beta-carotene are known in the art and can be applied to the formulation of oxidatively transformed carotenoids. See, for example, U.S. Patent Publication No. 20060182863, PCT Publication No. WO2008/110225, and Triplett et al., Journal of Nanoparticle Research 11:601 (2008), each of which is incorporated herein by reference.

Oxidatively transformed carotenoid particles can also be prepared by microencapsulation of the oxidatively transformed carotenoid, or a fractionated component thereof, in an encasing matrix which is microparticulate or nanoparticulate in size. The encasing matrices are non-toxic, impermeable and are biodegradable upon uptake into the target species. Microencapsulation techniques generally involve the coating of small solid particles or liquid droplets with a thin film of a material, the material providing a protective encasing matrix shell for the contents of the microcapsule. Typical encasing matrix materials may include, but are not limited to, gum arabic, gelatin, ethylcellulose, polyurea, polyamide, aminoplasts, maltodextrins, and hydrogenated vegetable oil. Typically the encasing matrix material includes a protein or a carbohydrate. Microencapsulation can be carried out using any of a variety of techniques known in the art, such as spray drying and extrusion. Spray drying is one of the most widely used methods of microencapsulation (i.e., dispersion and atomization). Extrusion is an encapsulation method in which a core material is dispersed in an amorphous mass of coating material and formed into microparticle (see U.S. Pat. No. 4,230,687). For example, the oxidatively transformed carotenoid, or a fractionated component thereof, can be encased in a matrix formed from a mixture of gelatin and sucrose to produce 5 μm size particles as described by Bo et al., Journal of Food Engineering 76:664 (2006). Alternatively, an encasing matrix formed from maltodextrin can be used as described by Saénz et al., Food Chemistry, 114:616 (2009).

The following examples are put forth so as to provide those of ordinary skill in the art with a complete disclosure and description of how the methods and compositions claimed herein are performed, made, and evaluated, and are intended to be purely exemplary of the invention and are not intended to limit the scope of what the inventors regard as their invention.

### EXPERIMENTAL METHODS

### Example 1

### Compound Preparation

OxBC is prepared from β-carotene (see U.S. Pat. No. 5,475,006) and stored at −20° C. prior to use. Stock solutions (50 mM of carotene equivalents—defined by the MW of β-carotene and the amount of β-carotene used to make the OxBC) are prepared by dissolving 26.85 mg OxBC/ml DMSO and stored as 500 μl aliquots at −80° C. Working 200 μM solutions of OxBC are prepared by dilution in the appropriate culture media and sterilized by filtration (0.22 μm pore size). The equivalent values of OxBC tested and the associated amount of DMSO in both test and control samples are indicated in Table 1. Equivalent amounts of DMSO vehicle are used as controls.

TABLE 1
Concentrations of OxBC and associated DMSO values

| OxBC (μM) | OxBC (μg/ml) | DMSO (%, v/v) |
|---|---|---|
| 0.0 | 0.00 | 0.000 |
| 0.1 | 0.05 | 0.001 |
| 0.5 | 0.27 | 0.005 |
| 1.0 | 0.54 | 0.010 |
| 2.5 | 1.34 | 0.025 |
| 5.0 | 2.67 | 0.050 |
| 10 | 5.38 | 0.100 |
| 15 | 8.01 | 0.150 |
| 25 | 13.4 | 0.250 |
| 50 | 26.9 | 0.500 |

### Example 2

### In Vitro Evaluation of OxBC Immune Modulating Activity

Zebrafish Embryonic Fibroblasts

Fibroblasts are a ubiquitous cell type present in most tissues. Although they are not considered as primary effectors of the innate immune system fibroblast do play a secondary role in tissue immunity. In our initial experiments in zebrafish fibroblasts (ZEM2S) it is shown that OxBC can induce increased expression of the homologous immune receptors in a fish species using quantitative-real-time PCR. An expanded panel of pathogen associated molecular pattern receptors (PAMPRs) is also assessed. OxBC is also evaluated for the ability to modulate cytokine production in fibroblasts under both naïve and pathogen challenge scenarios. Cytokines to be assayed include tumor necrosis factor alpha (TNFα), interleukin-1β (IL-1β), interleukin-6 (IL-6), interleukin-8 (IL-8), and monocyte chemotactic protein-1 (MCP-1).

Rainbow Trout Primary Head-Kidney Macrophages

In fish the head-kidney or pronephros represents the major hemopoetic tissue rich in cells of the monocyte-macrophage lineage. Macrophages along with neutrophils are the primary effector cell types of the innate immune system and methods for macrophage isolation from rainbow trout head-kidney are well established in the literature. Cultured primary monocytes/macrophages isolated from the rainbow trout head-kidney are used as a model system to evaluate OxBC's ability to modulate innate immune effectors in fish. OxBC-effects on immune parameters, such as phagocytic activity, cellular adherence, and cytokine/chemokine expression profiles, are measured. An expanded panel of PAMPRs, macrophage oxidative burst activity, and expression of antimicrobial enzymes and peptides, such as lysozyme and hepcidin, are also monitored.

Rainbow Trout Gill Epithelial Cell Line

The commercially available rainbow trout gill epithelial cell line (RTgill-W1) offers a suitable model for evaluating OxBC's ability to enhance epithelial immunity in fish. OxBC's effect on RTgill-W1 cells is assessed by measuring a panel of immune markers similar to that outlined above for the primary macrophages and zebrafish fibroblasts. In addition, OxBC's effect on epithelial barrier function is evaluated using the transepithelial transport assay.

### Example 3

### In Vivo Evaluation of OxBC Immune Modulating Activity

Embryonic Zebrafish

OxBC effects on embryonic zebrafish are assessed by culturing zebrafish embryos in the presence of OxBC. Embryos of various developmental stages are cultured in a 96-well plate and a range of OxBC concentrations will be assayed. Embryo viability, development, and immunity will be assessed. OxBC is also assessed using the alternative method of injection directly into the yolk sac. Because embryonic zebrafish obtain all of their nutritional requirements from the yolk the yolk sac injection method is akin to oral gavage in rodents and offers the advantage of individual dosing.

Adult Zebrafish

OxBC's ability to enhance immunity and increase resistance to infections in adult fish is assessed with feed trials in adult zebrafish to establish the effective dose range. The alternative mode of administering OxBC directly to the water, where it can be absorbed across the gills, is also evaluated. Initial trials are conducted in the absence of a pathogen challenge. In subsequent trials OxBC-effects on fish health are determined in challenge models with zebrafish exposed to specific bacterial pathogens.

Adult Rainbow Trout

OxBC's effects on rainbow trout in vitro models are evaluated in feed trials in adult trout. These studies can assess the ability of OxBC administered through feed to enhance innate immunity at the level of the gut, gill, and head-kidney macrophage. These trials can provide additional evidence of OxBC's efficacy to enhance immunity, health and performance in a fish species of relevance to the food-fish aquaculture sector.

### Example 4

### In Vitro Evaluation of OxBC Immune Modulating Activity in Primary Trout Leukocyte Cultures

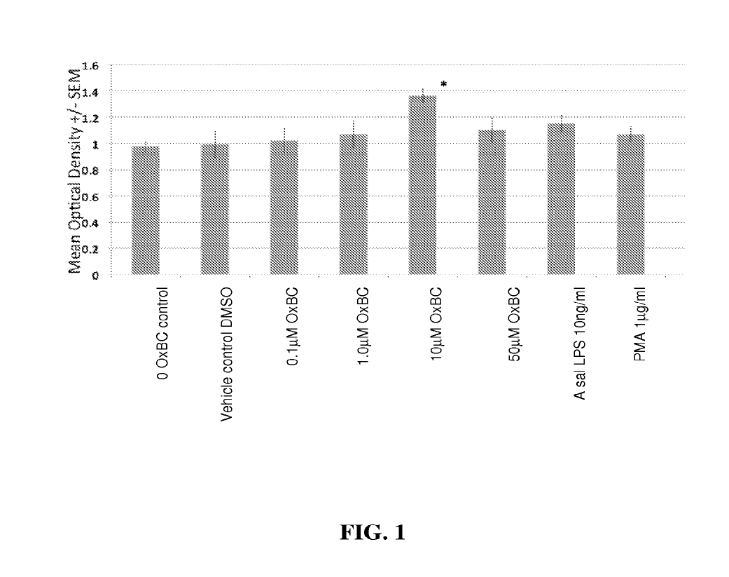

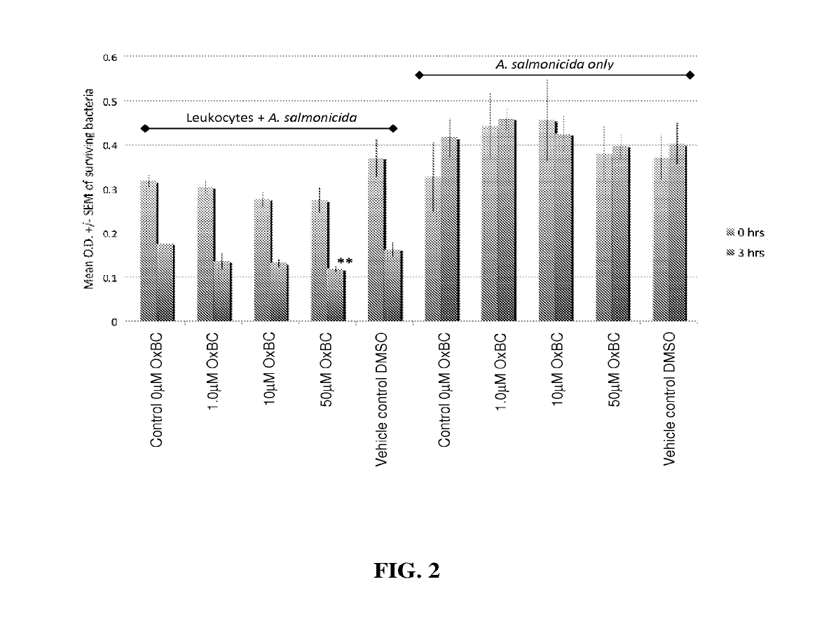

Experiments were conducted to determine the effect of OxBC in primary trout leukocyte cultures. These data are provided in FIGS. 1 and 2. Treatment with OxBC increased trout leukocyte respiratory burst activity by approximately 37% (FIG. 1) and bactericidal activity was increased by approximately 33% (FIG. 2). Together these results demonstrate the ability of OxBC to modulate the activity of cells that act as the primary effectors of innate immunity in fish. Respiratory burst and bactericidal activity are critical innate immune mechanisms used to defend the host against infection by bacterial pathogens. OxBC's ability to enhance these mechanisms in fish cells is proof of concept for the application of OxBC to enhance innate immunity in fish thereby helping to prevent or reduce the frequency and/or severity of bacterial infection in fish.

### Other Embodiments

All publications and patent applications, and patents mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims.

What we claim is:

1. A method of ameliorating an effect of physical stress in a fish, said method comprising administering to said fish a composition comprising oxidatively transformed carotenoid, or a fractionated component thereof, in an amount sufficient to ameliorate said effect of physical stress, wherein said physical stress arises from overcrowding or from an environmental change.

2. The method of claim 1, wherein said physical stress arises from changes in salinity, temperature, pH, oxidative stress, or exposure to a chemotherapeutic agent.

3. A method of ameliorating an effect of physical stress in a shellfish, said method comprising administering to said shellfish a composition comprising oxidatively transformed carotenoid, or a fractionated component thereof, in an amount sufficient to ameliorate said effect of physical stress.

4. The method of claim 3, wherein said physical stress arises from overcrowding.

5. The method of claim 3, wherein said physical stress arises from an environmental change.

6. The method of claim 5, wherein said physical stress arises from changes in salinity, temperature, pH, oxidative stress, or exposure to a chemotherapeutic agent.

7. The method of any of claims 1-6, wherein said effect of physical stress is concomitant with aggression.

8. The method of any of claims 1-6, wherein said effect of physical stress concomitant with a decrease in meat quality or marketability.

9. The method of any of claims 1-6, wherein said effect of physical stress is concomitant with an increase in mortality.

10. The method of claim 3, wherein said physical stress is not the result of an inflammatory condition or infection.

11. A method of treating a shellfish having, or at risk of, an infection, said method comprising administering to said shellfish oxidatively transformed carotenoid, or a fractionated component thereof, in an amount sufficient to treat said infection.

12. The method of claim 11, wherein said infection is by a bacterium, virus, fungus, protozoan, or parasite.

13. The method of claim 12, wherein said infection is a bacterial infection by bacillus, edwardsiella, renibacterium, flavobacterium, aeromonas, mycobacterium, haemophilus, nocardia, pasteurella, pseudomonas, streptococcus, yersinia, or vibrio spp.; a protozoan infection by amoeba, coccidia, ichthyoptheria, gregarina, or microspora spp.; or a viral infection selected from white spot, infectious pancreatic necrosis, viral hemorrhagic septicemia, infectious hematopoetic necrosis, and spring viremia.

14. A method of treating a shellfish having, or at risk of, an inflammatory condition, said method comprising administering to said shellfish a composition comprising oxidatively transformed carotenoid, or a fractionated component thereof, in an amount sufficient to treat said inflammatory condition.

15. The method of any of claims 3-14, wherein said shellfish is a crustacean selected from shrimp, prawn, lobster, crayfish, and crabs.

16. The method of any of claims 3-14, wherein said shellfish is a mollusk selected from clams, mussels, oysters, winkles, scallops, and squid.

17. The method of claim 1, wherein said fish is selected from catfish, carp, trout, salmon, char, whitefish, sturgeon, tench, roach, pike, pike-perch, sole, turbot, yellowtail, bass, milkfish, tilapia, walleye, gray mullet, eels, angel fish, barb, catfish, cichlids, corydoras, danio, discus, gourami, guppy, koi, loach, minnow, molly, platy, plecostumas, rainbow and platy variatus, rasbora, shark, sword, tetra, botia, knife fish, lionfish, brackish-archer fish, flounder, golby, half beak, mono, needle fish, pipe fish, puffer, scat, bumble bee, twin spot damsel, yellowtail damsel, barbed squirrel, wrasse, black-spotted puffer, trigger fish, puffer, and butterfly fish.

18. A method of treating a crustacean having, or at risk of, shell disease, said method comprising administering to said crustacean oxidatively transformed carotenoid, or a fractionated component thereof, in an amount sufficient to treat said shell disease.

19. The method of any of claims 1-18, wherein said administering comprises oral administration of a feed comprising oxidatively transformed carotenoid, or a fractionated component thereof.

20. The method of any of claims 1-18, wherein said administering comprises parenteral administration of a bath comprising oxidatively transformed carotenoid, or a fractionated component thereof.

21. The method of claim 20, wherein said parenteral administration comprises administration across a gill.

22. The method of any of claims 1-21, wherein said composition comprises fractionated oxidatively transformed carotenoid.

23. The method of any of claims 1-21, wherein said composition comprises unfractionated oxidatively transformed carotenoid.

24. A method of administering oxidatively transformed carotenoid, or a fractionated component thereof, to an aquatic animal, said method comprising suspending said oxidatively transformed carotenoid, or a fractionated component thereof, in an aqueous solution and immersing said aquatic animal in said aqueous solution.

25. The method of claim 24, wherein said oxidatively transformed carotenoid, or a fractionated component thereof, is formulated as a microemulsion.

26. The method of claim 24, wherein said oxidatively transformed carotenoid, or a fractionated component thereof, is formulated as a microparticle.

27. The method of claim 24, wherein said oxidatively transformed carotenoid, or a fractionated component thereof, is formulated as a suspension sized for administration across the gill of said aquatic animal.

28. The method of any of claims 24-27, wherein said aquatic animal is immersed in a bath, dip, flush, or indefinite bath.

29. The method of any of claims 24-28, wherein said aqueous solution comprises from 0.00001% to 0.05% (w/w) oxidatively transformed carotenoid, or a fractionated component thereof.

30. The method of any of claims 24-29, wherein the step of immersing is performed from 24 hours preceding to 24 hours following subjecting said aquatic animal to a physical stress.

31. The method of claim 30, wherein said physical stress arises from overcrowding.

32. The method of claim 30, wherein said physical stress arises from moving, netting, crowding, cleaning, counting, tagging, or transporting said aquatic animal.

33. The method of claim 30, wherein said physical stress arises from an environmental change.

34. The method of claim 33, wherein said physical stress arises from changes in salinity, temperature, pH, oxidative stress, or exposure to a chemotherapeutic agent.

35. The method of any of claims 24-34, further comprising oral administration of a feed comprising oxidatively transformed carotenoid, or a fractionated component thereof.

36. The method of any of claims 24-29, wherein the step of immersing is performed to treat an infection in said aquatic animal.

37. The method of claim 36, wherein said infection is a bacterial infection by bacillus, edwardsiella, renibacterium, flavobacterium, aeromonas, mycobacterium, haemophilus, nocardia, pasteurella, pseudomonas, streptococcus, yersinia, or vibrio spp.; a protozoan infection by amoeba, coccidia, ichthyoptheria, gregarina, or microspora spp.; or a viral infection selected from white spot, infectious pancreatic necrosis, infectious salmon anemia, viral hemorrhagic septicemia, infectious hematopoetic necrosis, and spring viremia.

38. The method of any of claims 24-29, wherein the step of immersing is performed to treat an inflammatory condition in said aquatic animal.

39. The method of any of claims 24-38, wherein said aqueous solution further comprises an anesthetic, antibiotic, or parasiticide.

40. The method of claim 39, wherein said aqueous solution further comprises an anesthetic selected from eugenol and tricaine methanesulfonate.

41. The method of claim 39, wherein said aqueous solution further comprises an antibiotic selected from oxytetracycline, florfenicol, amikacin, ceftazidime, enrofloxacin, nitrofurazone, and trimethoprim sulfadiazine.

42. The method of claim 39, wherein said aqueous solution further comprises a parasiticide selected from diflubenzuron, fenbendazole, formaldehyde, levamisole phosphate, metronidazole, praziquantel, and trichlorfon.

43. The method of any of claims 24-42, wherein said aquatic animal is a fish.

44. The method of claim 43, wherein said fish is selected from catfish, carp, trout, salmon, char, whitefish, sturgeon, tench, roach, pike, pike-perch, sole, turbot, yellowtail, bass, milkfish, tilapia, walleye, gray mullet, eels, angel fish, barb, catfish, cichlids, corydoras, danio, discus, gourami, guppy, koi, loath, minnow, molly, platy, Plecostumas, rainbow and platy variatus, rasbora, shark, sword, tetra, botia, knife fish, lionfish, brackish-archer fish, flounder, golby, half beak, mono, needle fish, pipe fish, puffer, scat, bumble bee, twin spot damsel, yellowtail damsel, barbed squirrel, wrasse, black-spotted puffer, trigger fish, puffer, and butterfly fish.

45. The method of any of claims 23-42, wherein said aquatic animal is a shellfish.

46. The method of claim 45, wherein said shellfish is a crustacean selected from shrimp, prawn, lobster, crayfish, and crabs.

47. The method of claim 45, wherein said shellfish is a mollusk selected from clams, mussels, oysters, winkles, scallops, and squid.

48. A mollusk feed comprising from 0.00001% to 0.005% (w/w) oxidatively transformed carotenoid, or a fractionated component thereof.

49. A crustacean feed comprising from 0.00001% to 0.005% (w/w) oxidatively transformed carotenoid, or a fractionated component thereof.

50. The crustacean feed of claim 49, further comprising from 10% to 60% (w/w) shellfish meal.

51. The crustacean feed of claim 49, wherein said crustacean feed is in the form of a crumble for use as a starter feed.

52. The crustacean feed of claim 49, wherein said crustacean feed is in the form of a pellet or flake for use as a grower feed or a finisher feed.

53. A composition comprising a mixture of oxidatively transformed carotenoid, or a fractionated component thereof, said composition having an effective particle size of from 20 nm to 10 μm.

54. The composition of claim 53, wherein said composition has an effective particle size of from 1 μm to 10 μm.

55. The composition of claim 53, wherein said composition has an effective particle size of from 20 nm to 1 μm.

56. The composition of claim 55, wherein said composition has an effective particle size of from 50 nm to 700 nm.

57. The composition of any of claims 53-56, wherein said oxidatively transformed carotenoid, or a fractionated component thereof, is encapsulated in an encasing matrix.

58. The composition of claim 57, wherein said encasing matrix comprises a protein or a carbohydrate.

59. A kit, comprising:
(i) a composition of any of claims 53-58; and
(ii) instructions for (a) mixing said composition with an aqueous solution and (b) immersing an aquatic animal in the aqueous solution.

60. The kit of claim 59, further comprising instructions for immersing said aquatic animal in a bath, dip, flush, or indefinite bath.

61. A kit, comprising:
(i) a feed comprising from 0.00001% to 0.005% (w/w) oxidatively transformed carotenoid, or a fractionated component thereof; and
(ii) instructions for administering said feed to a mollusk.

62. A kit, comprising:
(i) a feed comprising from 0.00001% to 0.005% (w/w) oxidatively transformed carotenoid, or a fractionated component thereof; and
(ii) instructions for administering said feed to a crustacean.
